(19) United States
(12) Patent Application Publication
(10) Pub. No.: US 2016/0213878 A1
(43) Pub. Date: Jul. 28, 2016

(54) Support Device for Air Tube

(71) Applicant: Robert Browning, JR., Paxton, MA (US)

(72) Inventor: Robert Browning, JR., Paxton, MA (US)

(21) Appl. No.: 15/005,155

(22) Filed: Jan. 25, 2016

Related U.S. Application Data
(60) Provisional application No. 62/106,831, filed on Jan. 23, 2015.

(51) Int. Cl. A61M 16/06; A61M 16/00

(52) U.S. Cl. CPC A61M 16/0683; A61M 16/0622; A61M 16/0057

(57) ABSTRACT

A support device for the hose of a continuous positive airway pressure (CPAP) machine. The support device includes an elongated strap that can be wrapped around the user's torso. A flexible tube is secured to the elongated strap, wherein the lower end of the tube is open and the upper end of the tube includes a facial mask thereon. The hose of a CPAP machine can be connected to the lower end of the flexible tube so that air flows through the flexible tube and into the facial mask. The facial mask further includes a head strap thereon that can be disposed around the user's head in order to secure the facial mask in the proper position. Thus, the support device allows a user to comfortably and securely utilize a CPAP machine that minimizes the risk of the hose becoming dislodged during sleep.

### CROSS REFERENCE TO RELATED APPLICATION

This application claims the benefit of U.S. Provisional Application No. 62/106,831 filed on Jan. 23, 2015. The above identified patent application is herein incorporated by reference in its entirety to provide continuity of disclosure.

### BACKGROUND OF THE INVENTION

1. Field of the Invention

The present invention relates to a support device. More specifically, the present invention provides a support device for use with a continuous positive airway pressure (CPAP) machine. The support device comprises an elongated strap adapted to be wrapped around the user's torso, wherein the elongated strap includes a tube mounted thereon. The lower end of the tube can be connected to the hose of a CPAP machine, and the upper end of the tube includes a facial mask thereon through which air can flow into the user's nose.

Sleep apnea is a common sleeping disorder wherein those afflicted suffer from pauses in breathing or periods of shallow breathing while sleeping. These occurrences are the result of the upper airway narrowing as the muscles relax during sleep, which reduces oxygen in the blood and causes a person to wake from sleep. Specifically, carbon dioxide builds up in the person's bloodstream causing the person to be aroused from sleep. As a result, the person may suffer from daytime sleepiness, slowed reaction time, and vision problems. People may also experience behavioral effects such as moodiness, decreased attentiveness, and decreased drive.

Moderate or severe sleep apnea is often treated using a continuous positive airway pressure (CPAP) device. CPAP machines are adapted to deliver a stream of compressed air in order to keep the airway open under pressure. This helps to prevent the airway from narrowing, allowing the person to sleep. CPAP machines require a facial mask to be worn by the user so that air can be provided through a hose from the CPAP machine into the facial mask. The hose often is unsupported, which can be particularly problematic as the hose can become twisted or tangled during sleep. Further, the hose may become dislodged, preventing air from passing to the facial mask. As a result, a support device for securing the tube of a CPAP machine on the user's body is desired.

In light of the devices known in the prior art, it is submitted that the present invention substantially diverges in design elements from the prior art and consequently it is clear that there is a need in the art for an improvement to existing tube support devices. In this regard the instant invention substantially fulfills these needs.

### SUMMARY OF THE INVENTION

In view of the foregoing disadvantages inherent in the known types of support devices now present in the prior art, the present invention provides a new support device wherein the same can be utilized for providing convenience for the user when supporting the hose of a CPAP machine so that the hose does not become twisted, tangled, or dislodged during sleep.

It is therefore an object of the present invention to provide a new and improved support device comprising an elongated strap adapted to be disposed around a user's torso, wherein the elongated strap supports a flexible tube thereon having a lower end and an upper end. The lower end is removably securable to the hose of a CPAP machine and the upper end includes a facial mask through which air can be delivered to the user's nose.

It is another object of the present invention to provide a support device having an elongated strap with a first end and a second end, wherein the elongated strap can be removably disposed around a user's torso by overlapping the first and second ends and securing the fasteners thereon.

Another object of the present invention is to provide a support device comprising an elongated strap having a flexible tube thereon, wherein the lower end of the tube is connectable to the hose of a CPAP machine via press fit, frictional fit, snap fit, or other securement means so that air can be delivered therethrough.

Yet another object of the present invention is to provide a support device comprising a facial mask through which air can flow, wherein the facial mask further includes a head strap for securing the facial mask in the proper position on the user's face, and also includes padding for providing comfort around the nasal region.

Another object of the present invention is to provide a support device that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 displays a perspective view of the support device.

FIG. 2 displays a perspective view of the support device as worn by a user.

### DETAILED DESCRIPTION OF THE INVENTION

Reference is made herein to the attached drawings. Like reference numerals are used throughout the drawings to depict like or similar elements of the support device. For the purposes of presenting a brief and clear description of the present invention, the preferred embodiment will be discussed as used for supporting the hose of a CPAP machine so that the tube does not become tangled, twisted, or dislodged while the person utilizing the device is sleeping. The figures are intended for representative purposes only and should not be considered to be limiting in any respect.

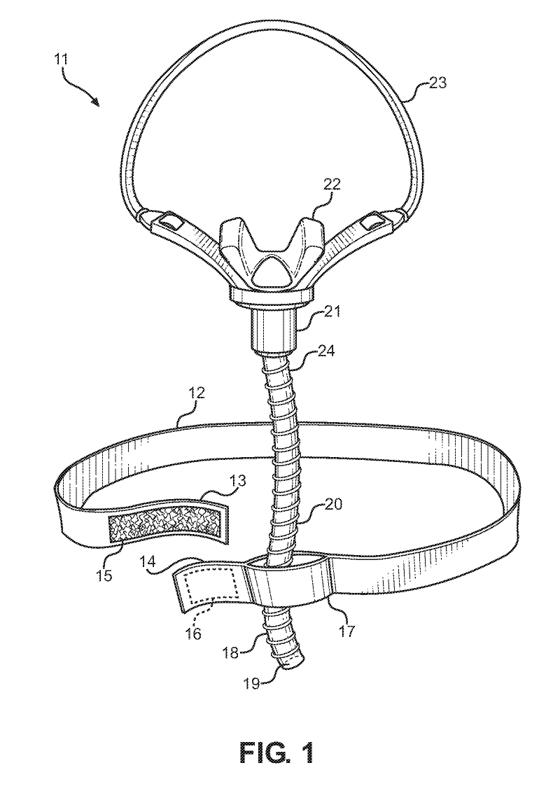

Referring now to FIG. 1, there is shown a perspective view of the support device. The support device 11 comprises an elongated strap 12 adapted to be wrapped around the torso of a user. The elongated strap 12 includes a first end 13 and a second end 14. The first end 13 includes a fastener 15 thereon and the second end 14 includes a mating fastener 16, such that the first and second ends 13, 14 can be adjustably secured together in an overlapping configuration. Preferably, the fastener 15 and mating fastener 16 comprise hook and loop fastening material. However, in alternate embodiments, the fastener 15 and mating fastener 16 can include other conventional fasteners such as buttons or snaps, among others.

A flexible tube 20 having an upper end 24 and a lower end 18 is affixed to the elongated strap 12. In the illustrated embodiment, the elongated strap 12 comprises a loop 17 through which the flexible tube 20 is positioned and secured. Preferably, the flexible tube 20 is positioned such that it is substantially orthogonal to the elongated strap 12. The lower end 18 includes an opening 19 on the terminal end thereof, wherein flexible tube 20 terminates adjacent to the elongated strap 12. The lower end 18 of the flexible tube 20 is adapted to be removably secured to the hose of a CPAP machine so that air can be delivered therethrough. The flexible tube 20 can be connected to the hose of a CPAP machine via press fit, threaded connection, snap connection, or other fitting method.

The upper end 24 of the flexible tube 20 includes a facial mask 21 thereon. The facial mask 21 is adapted to provide airflow to the user's nose and the facial mask 21 is shaped and sized so as to be positioned over a user's nose. In this way, airflow is provided through the flexible tube 20 through the facial mask 21 and into the user's nasal passages. The facial mask 21 is adapted to fit snugly onto the user's face. The facial mask 21 preferably includes padding 22 or cushioning thereon to provide comfort to the user around the nasal region.

A head strap 23 is affixed to the facial mask 21, wherein the head strap 23 can be wrapped around a user's head in order to position and secure the facial mask 21 on the user's face. The head strap 23 is preferably composed of a stretchable or elastic material so that the head strap 23 can fit snugly onto the head of various users. In alternate embodiments, the head strap 23 includes an adjustable fastener thereon to adjust the size thereof in order to provide a secure fit on the user's head.

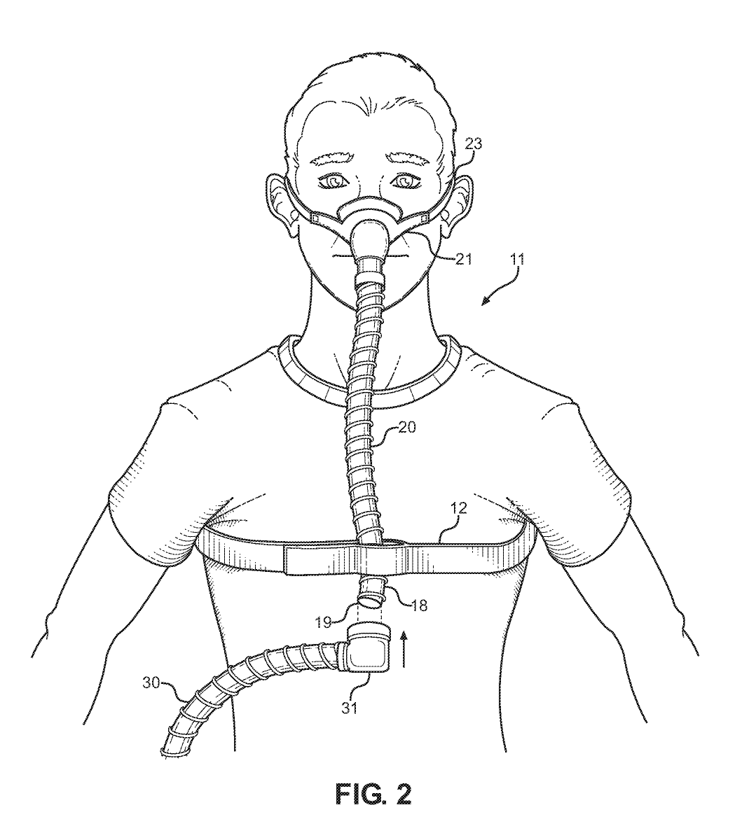

Referring now to FIG. 2, there is shown a perspective view of the support device as worn by a user. In operation, the user can wrap the elongated strap 12 around his or her torso and can secure the first and second ends in an overlapping fashion via the mating fasteners thereon. The user can position the facial mask 21 over his or her nose and can dispose the head strap 23 around the user's head in order to secure the facial mask 21 in the desired position. The flexible tube 20 extends from the elongated strap 12 to the facial mask 21. The flexible tube 20 can bend and flex in order to accommodate the movement of the user during sleep.

Once the support device 11 is positioned on the user, the user can connect the hose 30 of a CPAP machine to the opening 19 at the lower end 18 of the flexible tube 20. The first end 31 of the hose 30 of the CPAP machine can be connected to the lower end 18 of the flexible tube 20 via press fit, snap connection, threaded connection, or any of various conventional securement means. In this way, airflow travels through the hose 30 of the CPAP machine and through the flexible tube 20, into the facial mask 21, and into the user's nose.

In the illustrated configuration, the hose 30 of the CPAP machine extends outward from the side of the user's torso and is less likely to become dislodged or twisted while the user is sleeping. Further, the flexible tube 20 is positioned so that it is maintained close to the user's body so that it does not become tangled, twisted, or dislodged while the user moves or changes positions while sleeping.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A support device, comprising:
an elongated strap comprising a first end and a second end, wherein the first end and second end are removably securable to one another, and wherein said elongated strap is adapted to be secured about a user's torso;
a tube having an upper end and a lower end, wherein said tube is affixed to said elongated strap;
said lower end adapted to be removably secured to a hose of a continuous positive airway pressure machine;
said upper end having a facial mask thereon through which air can flow.

2) The support device of claim 1, wherein said tube is flexible.

3) The support device of claim 1, wherein said facial mask comprises a head strap thereon adapted to secure the facial mask on the user's head.

4) The support device of claim 1, wherein said first end and said second end of said elongated strap include hook and loop fastening material thereon.

5) The support device of claim 1, wherein said tube is permanently affixed to said elongated strap.

6) The support device of claim 1, wherein said facial mask comprises padding thereon adapted to be positioned against a user's face.
